(19) United States
(12) Patent Application Publication
(10) Pub. No.: US 2021/0247337 A1
(43) Pub. Date: Aug. 12, 2021

(54) METHOD FOR DETERMINING SOFTENING TEMPERATURE OF COPPER AND COPPER ALLOY

(71) Applicant: Shanghai Electric Cable Research Institute Co., Ltd., Shanghai (CN)

(72) Inventors: Dawei YAO, Shanghai (CN); Yuanwang ZHANG, Shanghai (CN); Shusen WANG, Shanghai (CN)

(73) Assignee: Shanghai Electric Cable Research Institute Co., Ltd., Shanghai (CN)

(21) Appl. No.: 17/256,012

(22) PCT Filed: Jun. 18, 2019
(86) PCT No.: PCT/CN2019/091705

§ 371 Date: Dec. 24, 2020

(30) Foreign Application Priority Data
Jul. 2, 2018 (CN) 201810707573.6

(51) Int. Cl. G01N 25/04; G01N 3/18; G01N 33/20

(52) U.S. Cl. CPC G01N 25/04; G01N 3/18; G01N 2203/0298; G01N 2203/0017; G01N 33/20

(57) ABSTRACT

The present disclosure relates to a method for determining a softening temperature of copper and copper alloy, including: selecting a plurality of samples of the same material, annealing the plurality of samples at different temperatures, air-cooling the plurality of samples; measuring the tensile strength of an original sample and the plurality of samples after the annealing; plotting a data of the measured tensile strength into a temperature-tensile strength curve; a temperature at which the tensile strength in the temperature-tensile strength curve drops to a certain value of an original sample tensile strength is the softening temperature of the material. The above technical solution in the present disclosure uses the tensile strength instead of hardness to measure the softening temperature. It can effectively improve the detection efficiency and reduce the cost, and can be widely used in determination of the softening temperature of fine materials of copper and copper alloy.

### CROSS REFERENCE TO RELATED APPLICATION

This is a Sect. 371 National Stage of PCT International Application No. PCT/CN2019/091705, filed on 18 Jun. 2019, which claims priority of a Chinese Patent Application No. 2018107075736 filed on 2 Jul. 2018, the contents of which hereby being incorporated by reference in its entirety for all purposes.

### BACKGROUND

### Field of Disclosure

The present disclosure relates to the field of metallic materials, in particular, to a method for determining a softening temperature of copper and copper alloy.

### Background

The processing technology of high-performance alloy conductive materials and its fine materials is indispensable in the advanced electronic material technology. The fine material formed of copper and copper alloy is the core material of the electronics industry and is the current focus of the basic material industry. As an important conductive and thermally conductive material for equipment, fine copper and copper alloys are widely used to connect key parts such as lead frames for semiconductor chips, transformers, braided wires, and microcomputer circuit boards. These applications require the fine copper and copper alloys to maintain stable performance at certain high temperatures, that is, to have a certain resistance to high temperature and softening. At present, a method for measuring the softening temperature of copper and copper alloys has been proposed, such as the GB/T 33370-2016 standard. However, this method is mainly applicable to large-size copper and copper alloy bars, pipes, plates, and other copper materials, whose hardness is easy to measure. However, for copper alloy fine wires, pipes, and strips with small dimensions and large deformations, on the one hand, the size is small and the hardness is difficult to measure, on the other hand, the unevenness of internal and external hardness caused by deformation processing and other factors makes the test results inaccurate. Besides, for the hardness measurement of these fine materials, the methods usually include inlaying, polishing, and hitting hardness, which greatly increases the actual workload and measurement cost. Therefore, it is particularly important to find a method to accurately determine the softening temperature of copper alloy fine materials.

### SUMMARY

The present disclosure prepares a method for determining a softening temperature of copper and copper alloy, to solve the problems in the art.

The present disclosure prepares a method for determining a softening temperature of copper and copper alloy. The determining method includes: selecting a plurality of samples of the same material, annealing the plurality of samples at different temperatures, and air-cooling the plurality of samples; measuring the tensile strength of an original sample and the plurality of samples after the annealing; and plotting a data of the measured tensile strength into a temperature-tensile strength curve; a temperature at which the tensile strength in the temperature-tensile strength curve drops to a certain value of an original sample tensile strength is the softening temperature of the sample.

More preferably, a temperature at which the tensile strength in the temperature-tensile strength curve drops to 80% of the original sample tensile strength is the softening temperature of the sample.

Preferably, the material includes copper and copper alloy material having a wire diameter of less than 1.024 mm or a thickness of less than 0.51 mm. It is generally called fine material of copper and copper alloy.

Preferably, the material is a wire, a foil, or a strip.

Preferably, the annealing is performed under vacuum or inert gas protection. When the annealing is performed under vacuum, the vacuum degree is less than 10⁻² Pa. When the soak annealing is performed under the protection of inert gas, the inert gas includes one or two of nitrogen or argon.

Preferably, the plurality of samples is annealed for 1 hour, the temperature of the soak annealing includes at least 10 different temperatures, and intervals of the 10 different temperatures are the same and are 10-20° C.

Preferably, when the material is a wire, the tensile strength is measured by clamping the wire on a fixture of a tensile testing machine, a clamping distance of the fixture is a gauge length, the gauge length of the wire ranges from 200 to 250 mm, and a tensile speed is 100 mm/min. More preferably, the average value of the tensile strength of the same wire sample obtained through three measurements is taken as the tensile strength of this wire.

Preferably, when the material is a foil or strip, the tensile strength is measured by: processing the sample into a dumbbell-shaped structure, a middle part of the dumbbell-shaped structure with a smaller cross section is a gauge length, and both ends of the dumbbell-shaped structure with a larger section are outside the gauge length; clamping the foil or strip on a fixture of a tensile testing machine, clamping the both ends of the dumbbell-shaped structure, a tensile speed is 100 mm/min. More preferably, a cross-sectional area of the middle part is set as S₀, when 11.3\sqrt{S₀}<10 mm, the gauge length is 10 mm; when 11.3\sqrt{S₀}≥10 mm the gauge length is 11.3\sqrt{S₀}. More preferably, the average value of the tensile strength of the same foil or strip sample obtained through three measurements is taken as the tensile strength of the foil or strip.

Preferably, when plotting the temperature-tensile strength curve, an interpolation method is used. The ordinate represents the tensile strength, and the abscissa represents the temperature of the soak annealing treatment.

For copper and copper alloys, the tensile strength and hardness have a certain linear relationship. The tensile strength and hardness can be converted to each other in a certain extent, because the process of testing the tensile strength and hardness is caused by the plastic deformation of the metal caused by stress, plastic deformation is achieved through slippage microscopically.

The above technical solution in the present disclosure uses the tensile strength instead of hardness to measure the softening temperature of fine materials of copper and copper alloy. It can effectively improve the detection efficiency and reduce the cost, and can be widely used in the determination of the softening temperature of fine materials of copper and copper alloy. Preferably, the fine material includes copper and copper alloy material having a wire diameter of less than 1.024 mm or a thickness of less than 0.51 mm.

### BRIEF DESCRIPTION OF THE DRAWINGS

FIG. 1 shows a temperature-tensile strength curve plotted according to Embodiment 1 of the present disclosure.

FIG. 2 shows a temperature-hardness curve plotted for Embodiment 1 with reference to a method in GB/T 33370-2016.

FIG. 3 shows a temperature-tensile strength curve plotted according to Embodiment 2 of the present disclosure.

FIG. 4 shows a temperature-tensile strength curve plotted according to Embodiment 3 of the present disclosure.

### DETAILED DESCRIPTION OF THE PREFERRED EMBODIMENTS

The embodiments of the present disclosure will be described below through exemplary embodiments. Those skilled in the art can easily understand other advantages and effects of the present disclosure according to contents disclosed by the specification. The present disclosure can also be implemented or applied through other different exemplary embodiments. Various modifications or changes can also be made to all details in the specification based on different points of view and applications without departing from the spirit of the present disclosure.

It should be noted that processing equipment or devices not specifically noted in the following embodiments are all conventional equipment or devices in the field.

Besides, it should be understood that one or more method operations mentioned in the present disclosure are not exclusive of other method operations that may exist before or after the combined operations or that other method operations may be inserted between these explicitly mentioned operations, unless otherwise stated. It should also be understood that the combined connection relationship between one or more equipment/devices mentioned in the present disclosure does not exclude that there may be other equipment/devices before or after the combined equipment/devices or that other equipment/devices may be inserted between these explicitly mentioned equipment/devices, unless otherwise stated. Moreover, unless otherwise stated, the numbering of each method step is only a convenient tool for identifying each method step, and is not intended to limit the order of each method step or to limit the scope of the present disclosure. The change or adjustment of the relative relationship shall also be regarded as the scope in which the present disclosure may be implemented without substantially changing the technical content.

### Embodiment 1

In this embodiment, the softening temperature of the copper alloy fine wire with a diameter of 0.05 mm was determined, which includes the following operations:

Eleven copper alloy wires with a diameter of 0.05 mm were used as samples, each with a length of 3 m, and the samples were subjected to vacuum annealing for an hour, in which the vacuum degree was 5.0×10⁻³ Pa, and the annealing temperatures were respectively 280° C., 300° C., 320° C., 340° C., 360° C., 380° C., 400° C., 420° C., 440° C., 460° C., 480° C., followed by air cooling. The air cooling means that the heated samples were directly placed in a room temperature environment to cool.

Tensile strength was measured and recorded on the samples without annealing and samples after annealing. The tensile gauge length was 200 mm and the tensile speed was 100 mm/min.

The measured data was plotted into a temperature-tensile strength curve. The abscissa represents the temperature of the annealing treatment, and the ordinate represents the tensile strength, referring to FIG. 1 for details.

The tensile strength of the samples without annealing (the original samples) was 390

MPa.

The temperature at which the strength of the material after the annealing treatment is reduced to 80% of the original sample strength is the softening temperature of the copper alloy fine wire with a diameter of 0.05 mm. That is, the softening temperature of the copper alloy fine wire was 351° C.

### Comparative Example of Embodiment 1

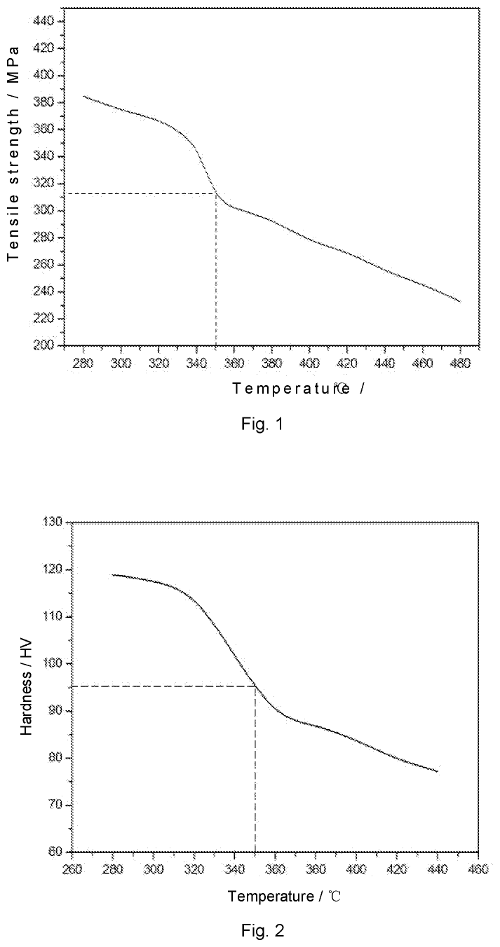

In this comparative example, the softening temperature of the copper alloy fine wire with a diameter of 0.05 mm in embodiment 1 was determined according to the method in GB/T 33370-2016. The softening temperature curve drawn by the measured results is shown in FIG. 2. The softening temperature of the copper alloy fine wire determined by this method was 350° C.

To conveniently show the advantages of the present disclosure over the method in GB/T 33370-2016, the original data obtained by the two methods are listed in Table 1.

TABLE 1

| | Tensile | Hard- | | Tensile | Hard- | | Tensile | Hard- |
|---|---|---|---|---|---|---|---|---|
| | strength | ness | | strength | ness | | strength | ness |
| Original | 388.74 | 115.97 | 280° C. | 384.44 | 119.74 | 300° C. | 374.51 | 116.71 |
| Sample | 389.59 | 118.97 | soaking | 385.42 | 118.21 | soaking | 374.59 | 117.46 |
| | 388.45 | 122.89 | for 1 h | 384.93 | 115.24 | for 1 h | 374.55 | 116.71 |
| | | 118.98 | | | 116.71 | | | 122.89 |
| | | 117.46 | | | 119.74 | | | 118.21 |
| | | 118.97 | | | 123.71 | | | 114.51 |
| | | 119.74 | | | 118.97 | | | 114.97 |
| | | 118.21 | | | 119.74 | | | 116.71 |
| 320° C. | 371.25 | 114.51 | 340° C. | 350.67 | 100.13 | 360° C. | 302.54 | 86.85 |
| soaking | 367.68 | 117.46 | soaking | 349.31 | 100.72 | soaking | 302.07 | 86.37 |
| for 1 h | 369.90 | 114.51 | for 1 h | 349.14 | 95.57 | for 1 h | 301.67 | 89.29 |
| | | 119.74 | | | 103.15 | | | 86.85 |
| | | 115.97 | | | 101.32 | | | 88.30 |
| | | 113.08 | | | 103.15 | | | 91.82 |
| | | 113.79 | | | 104.4 | | | 89.29 |
| | | 112.37 | | | 105.13 | | | 90.29 |
| 380° C. | 293.54 | 84.51 | 400° C. | 278.97 | 83.6 | 420° C. | 270.49 | 78.45 |
| soaking | 294.18 | 88.30 | soaking | 276.87 | 81.83 | soaking | 269.69 | 79.27 |
| for 1 h | 292.79 | 85.90 | for 1 h | 276.76 | 84.05 | for 1 h | 270.09 | 77.23 |
| | | 84.05 | | | 84.51 | | | 76.44 |
| | | 83.60 | | | 83.15 | | | 79.69 |
| | | 88.79 | | | 85.9 | | | 81.83 |
| | | 86.37 | | | 85.9 | | | 79.69 |
| | | 91.31 | | | 83.15 | | | 85.44 |
| 440° C. | 257.35 | 76.44 | 460° C. | 246.4441 | 78.04 | 480° C. | 229.94 | 70.16 |
| soaking | 255.28 | 78.45 | soaking | 245.78 | 74.5 | soaking | 231.2 | 67.79 |
| for 1 h | 255.57 | 77.23 | for 1 h | 245.39 | 73.38 | for 1 h | 230.98 | 70.85 |
| | | 77.23 | | | 75.27 | | | 71.92 |
| | | 77.63 | | | 74.88 | | | 77.63 |
| | | 78.04 | | | 76.44 | | | 70.85 |
| | | 78.45 | | | 74.13 | | | 70.5 |
| | | 76.44 | | | 73.75 | | | 67.79 |

For the softening temperature measured by the above two methods, the softening temperature measured by the method of the present disclosure was 351° C., and the softening temperature measured by the method in GB/T 33370-2016 was 350° C. The softening temperatures obtained by the two methods are the same. Analyzing the original data of the two methods, it is not difficult to find that the dispersion of the tensile strength data is small and the repeatability is good. The hardness data is more discrete, and it is easier to produce measurement errors than the tensile strength. Therefore, it can be considered that the softening temperature calculated by the tensile strength is more accurate.

Relevant professional technicians can clearly understand that for fine materials, the determination of tensile strength is much simpler than the determination of hardness.

In summary, the method for determining the softening temperature of copper and copper alloy prepared by the present disclosure has high accuracy and the operation method is simple.

### Embodiment 2

In this embodiment, the softening temperature of a copper alloy foil with a thickness of 0.09 mm was determined, which includes the following operations:

Fifteen sheets of copper alloy foil with a thickness of 0.09 mm were taken as samples, and the samples were respectively subjected to annealing for an hour under argon atmosphere. The soak annealing temperatures were respectively 250° C., 260° C., 270° C., 280° C., 290° C., 300° C., 310° C., 320° C., 330° C., 340° C., 350° C., 360° C., 380° C., 390° C., 400° C., followed by air cooling.

Tensile strength was measured and recorded on the samples without annealing and samples after annealing. The samples were processed into dumbbell-shaped structures. The tensile gauge length was 10 mm, and the tensile speed was 100 mm/min.

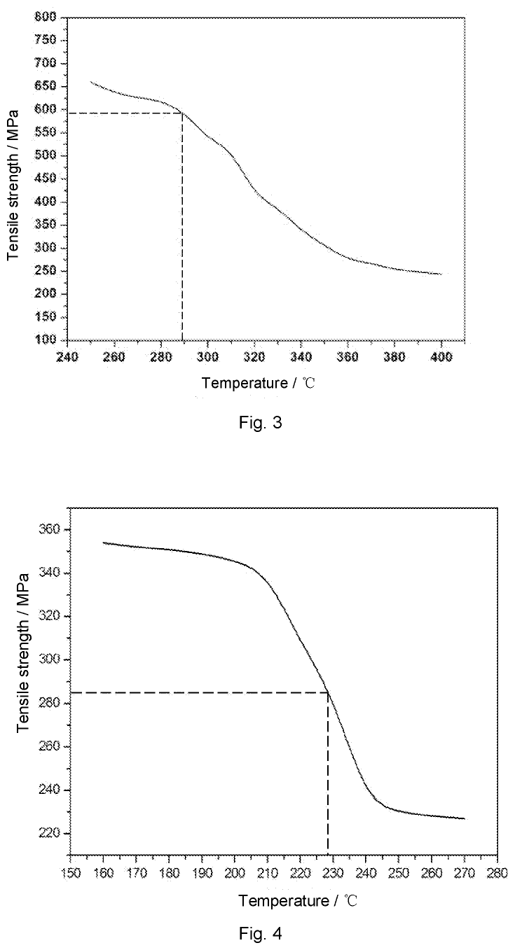

The measured data was plotted into a temperature-tensile strength curve. The abscissa represents the temperature of the annealing treatment, and the ordinate represents the tensile strength, referring to FIG. 3 for details.

The tensile strength of the samples without annealing (the original samples) was 740 MPa.

The temperature at which the strength of the material after the annealing treatment is reduced to 80% of the original sample strength is the softening temperature of the samples. The softening temperature of the copper alloy foil was determined to be 288° C.

### Embodiment 3

In this embodiment, the softening temperature of a pure copper strip with a thickness of 0.30 mm was determined, which includes the following operations:

Eleven samples were taken, and the samples were respectively subjected to annealing treatment for an hour under a nitrogen atmosphere. The soak annealing treatment temperatures were respectively 160° C., 170° C., 180° C., 190° C., 200° C., 210° C., 220° C., 230° C., 240° C., 250° C., and 260° C., followed by air cooling.

Tensile strength was measured and recorded on the samples without annealing treatment and the samples processor after annealing treatment. The samples were processed into dumbbell-shaped structures. The tensile gauge length was by the 10 mm, and the tensile speed was 100 mm/min.

The measured data was plotted into a temperature-tensile strength curve. The abscissa represents the temperature of the soak annealing treatment, and the ordinate represents the tensile strength, referring to FIG. 4 for details.

The tensile strength of the samples without annealing (the original samples) was 356 MPa.

The temperature at which the strength of the material after the soak annealing treatment is reduced to 80% of the original sample strength is the softening temperature of the samples. The softening temperature of the copper strip was determined to be 228° C.

The above-mentioned embodiments are merely illustrative of the principle and effects of the present disclosure instead of limiting the present disclosure. Modifications or variations of the above-described embodiments may be made by those skilled in the art without departing from the spirit and scope of the disclosure. Therefore, all equivalent modifications or changes made by those skilled in the art without departing from the spirit and scope of the disclosure will be covered by the appended claims.

1. A method for determining a softening temperature of copper and copper alloy, comprising:
selecting a plurality of samples of a same material, annealing the plurality of samples at different temperatures, and air-cooling the plurality of samples;
measuring a tensile strength of an original sample and the plurality of samples after the annealing; and
plotting a data of the measured tensile strength into a temperature-tensile strength curve;
a temperature at which the tensile strength in the temperature-tensile strength curve drops to a certain value of an original sample tensile strength is the softening temperature of the material.

2. The determining method according to claim 1, wherein the material includes copper and copper alloy material having a wire diameter of less than 1.024 mm or a thickness of less than 0.51 mm.

3. The determining method according to claim 1, wherein the material is a wire, a foil, or a strip.

4. The determining method according to claim 1, wherein the annealing is performed under vacuum or inert gas protection.

5. The determining method according to claim 1, wherein the temperature of the annealing includes at least 10 different temperatures, and intervals of the 10 different temperatures are the same and are 10-20° C.

6. The determining method according to claim 1, wherein when the material is a wire, the tensile strength is measured by clamping the wire on a fixture of a tensile testing machine, a clamping distance of the fixture is a gauge length, the gauge length of the wire ranges from 200 to 250 mm, and a tensile speed is 100 mm/min.

7. The determining method according to claim 1, wherein when the material is a foil or strip, the tensile strength is measured by:
processing the sample into a dumbbell-shaped structure, a middle part of the dumbbell-shaped structure with a smaller cross section is a gauge length, and both ends of the dumbbell-shaped structure with a larger section are outside the gauge length; and
clamping the foil or strip on a fixture of a tensile testing machine, clamping the both ends of the dumbbell-shaped structure, a tensile speed is 100 mm/min.

8. The determining method according to claim 7, wherein a cross-sectional area of the middle part is set as S₀, when 11.3\sqrt{S₀}<10 mm, the gauge length is 10 mm; when 11.3\sqrt{S₀}≥10 mm, the gauge length is 11.3\sqrt{S₀}.

9. Use of the method according to claim 1 for determining a softening temperature of fine material of copper and copper alloy.

10. The use according to claim 9, wherein the fine material includes copper and copper alloy material having a wire diameter of less than 1.024 mm or a thickness of less than 0.51 mm.

11. The determining method according to claim 1, wherein the plurality of samples is annealed for 1 hour.
